(19) United States
(12) Patent Application Publication
(10) Pub. No.: US 2004/0152091 A1
(43) Pub. Date: Aug. 5, 2004

(54) Biosensor, device and method for detecting nucleic acids by means of at least two units for immobilizing nucleic acids

(76) Inventors: Christian Paulus, Weilheim (DE); Roland Thewes, Grobenzell (DE); Meinard Schiente, Neubiberg (DE)

Correspondence Address: Jeffery R Stone, Briggs and Morgan, 2200 IDS Center, 80 South Eighth Street, Minneapolis, MN, 55402, US

(21) Appl. No.: 10/472,074

(22) PCT Filed: Mar. 12, 2002
(86) PCT No.: PCT/DE02/00867

(30) Foreign Application Priority Data
Mar. 16, 2001 (DE) 101 12 778.2

(51) Int. Cl. C12Q 1/68

(52) U.S. Cl. 435/6

(57) ABSTRACT

A device and method for detecting nucleic acids. The device having a biosensor with at least two nucleic acid immobilization units and an electrical detection circuit. In the biosensor, the at least two nucleic acid immobilization units are in this case electrically conductive and electrically insulated from one another. The at least two nucleic acid immobilization units are provided with first nucleic acid molecules acting as scavenger molecules. The first nucleic acid molecules are present as single-stranded molecules and can bind second nucleic acid molecules to be detected. The first single-stranded nucleic acid molecules acting as scavenger molecules are provided with a redox-active label capable of generating a detectable signal. The electrical detection circuit is configured in such a way that it detects the hybridization even of the nucleic acid molecules with the scavenger molecules by means of the label.

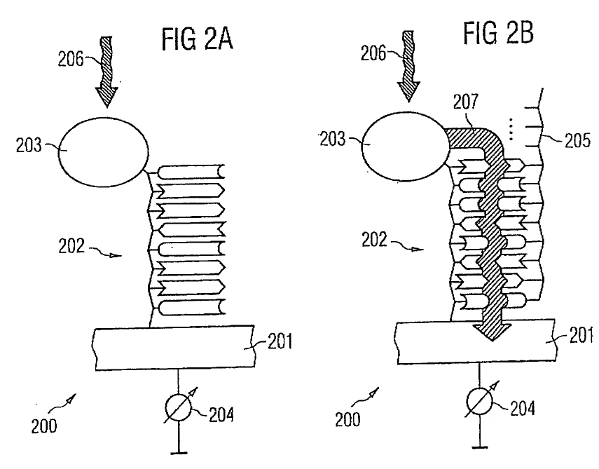

The invention relates to a biosensor, a device and a method for detecting nucleic acids by means of at least two nucleic acid immobilization units.

[1] to [5] disclose an electrochemical method for detecting nucleic acid-oligomer hybridization events, i.e. put more simply a method for detecting nucleic acids. This method, as explained in more detail in FIG. 2, is based on the difference in conductivity between single-stranded and double-stranded nucleic acids.

Furthermore, [6] discloses a device in which probe molecules, which can bind to a previously determined molecular target structure, are applied to test sites. In the device according to [6], after application of a signal, particular electrical, mechanical or optical properties of the test sites are used in order to detect the binding of the probe molecules with a target structure.

In addition, [7] discloses a device in which individually addressable micro-sites can be used to carry out molecular biological reactions such as nucleic acid hybridizations. The addressability of a micro-site is achieved by a DC micro-electrode arranged under it. The purpose of the latter is to provide electrophoretic attraction or repulsion of binding units or reactants and thus to control the molecular biological reaction.

FIGS. 2a and 2b schematically show the functional principle of the method according to [1] to [5]. A sensor 200 has a uniformly configured conductive surface layer 210, on which single-stranded nucleic acid/DNA molecules 202 are immobilized at particular positions. The single-stranded nucleic acid molecules 202 act as scavenger molecules for a nucleic acid to be detected, and they have a redox-active label 203. The surface layer 201 is furthermore provided with an electrical terminal 204.

The sensor 200 is brought in contact with a sample (not shown) to be studied, for example a liquid electrolyte.

If DNA strands 205 with a sequence which is complementary to the sequence of the DNA scavenger molecules 202 are contained in the electrolyte, then these DNA strands 205 hybridize with the DNA scavenger molecules 202 (cf. FIG. 2b).

Hybridization of a DNA scavenger molecule 202 and a DNA strand 205 takes place only if the sequences of the respective DNA probe molecule 202 and of the corresponding DNA strand 205 are complementary to one another. If this is not the case, then no hybridization takes place. A DNA probe molecule with a predetermined sequence is hence capable of binding, i.e. hybridizing with, only a particular DNA strand in each case, namely the one with the respective complementary sequence.

Double-stranded nucleic acid molecules are formed as a result of the hybridization, as can be seen from FIG. 2b.

In the method according to [1] to [5], after a rinsing step (not shown), light with a suitable wavelength is irradiated onto the sensor 200, as symbolized by the arrow 206. The redox-active label 203 continuously releases electrons when it is stimulated by the incident light. If the label is present in a double-stranded hybrid of a DNA scavenger molecule 202 and a nucleic acid 205 to be detected, this double-stranded hybrid acts as a kind of electron pump and conducts electrons from the label 203 to the conductive surface 201, as illustrated by the arrow 207, so that a current can be measured at the latter (cf. FIGS. 2b,c). If no hybridization takes place, however, single-stranded scavenger molecules 202 approximately constitute an insulator, so that no current flows on the surface 201.

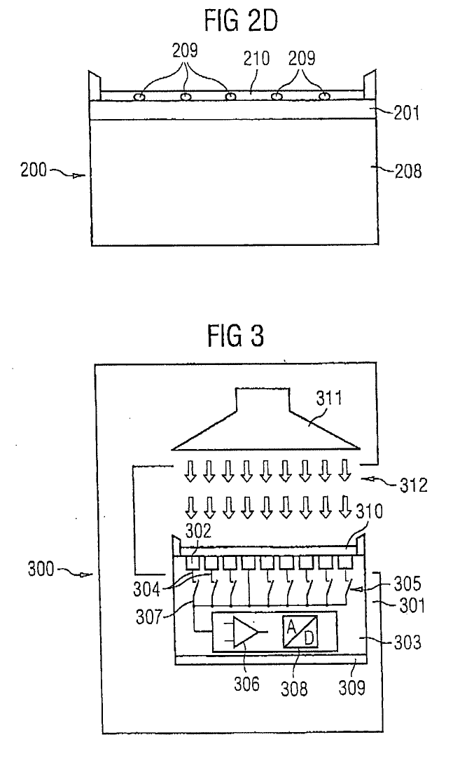

FIG. 2c, which corresponds to FIG. 4 from [3], shows a more accurate representation in molecular detail, in which a photosynthetic bacterial reaction center is used as the redox-active label.

In [1] to [5], the current flow caused by the double-stranded molecule is detected by a sensor 200 which is configured in such a way that a continuous electrically conductive gold film 201 is applied on an insulating substrate material 208, such as a glass wafer/muscovite wafer (cf. e.g. [3], page 20, section “Modified surfaces/electrodes and page 54, 4th paragraph, page 56, Example 2 and page 29, 1st paragraph). In this case, one or more so-called “test sites”, i.e. previously determined positions or sensor fields on which scavenger molecules are immobilized, are situated on the gold film. Such a sensor 200, which has a gold surface 201 formed surface-wide and provided with various test sites 209, and a substrate material 208, is shown in FIG. 2d. FIG. 2d furthermore shows an analyte 210 to be studied on the biosensor 200.

A sensor according to [1] to [5] hence operates as a purely passive arrangement, i.e. as a sensor, which must be connected to external measuring devices in order to detect/evaluate the signal.

It is evidently regarded as an advantage of such a sensor that the individual test sites do not need to be applied on individual (micro)-electrodes which are electrically insulated from one another and can be driven individually for application of a potential and for read-out (cf., for example [3], page 55, first paragraph). Rather, addressing of individual sites is carried out by controlled irradiation with light with a suitable wavelength.

Such a “passive” sensor with external evaluation units, however, seems disadvantageous for several reasons.

The surface-wide layer gives rise to a high capacitance, since all the sensor fields are constantly interconnected with the external electrical read-out unit. The signals which, in the method, are customarily small can be so burdened by this comparatively high load capacitance that their detection by means of an external current measuring instrument seems problematic, or at least takes place with very large time constants.

Dark currents, i.e. parasitic currents, can furthermore occur from positions not selected by light irradiation. These dark currents may even exceed the signal current, which can restrict the maximum possible number of sensor fields.

Furthermore, the crosstalk between individual sensor fields seems to be a problem in such arrays, especially when the currents to be detected are very small.

In the case of a passive, optically addressable chip, an external reading device has to process very small analog signals. This is susceptible to interference in an environment where there are further electrical devices.

Lastly, the accuracy with which the light is irradiated onto the sensor also seems problematic. The incident light must selectively strike the chosen individual sensor fields, but without striking the neighbor fields. This requires an elaborate optical arrangement for read-out of the sensor.

It is therefore an object of the present invention to provide an alternative method and an alternative biosensor and a device for detecting nucleic acids, which do not have these disadvantages.

The object is achieved by the method, the biosensor and the device with the features according to the independent patent claims.

The method for detecting nucleic acid uses at least two nucleic acid immobilization units. In this case, the at least two nucleic acid immobilization units are electrically conductive and electrically insulated from one another.

In the method, the at least two nucleic acid immobilization units are provided with first nucleic acid molecules acting as scavenger molecules. The first nucleic acid molecules are present as single-stranded molecules and can bind second nucleic acid molecules to be detected. Furthermore, the first single-stranded nucleic acid molecules acting as scavenger molecules are provided with a redox-active label which can generate a detectable signal.

The labeling may be carried out either before the scavenger molecules are immobilized on the at least two nucleic acid immobilization units or after the scavenger molecules are immobilized on the at least two nucleic acid immobilization units.

In the method, a sample to be studied is brought in contact with the at least two nucleic acid immobilization units, wherein the sample to be studied may contain the second nucleic acid molecules to be detected. Second nucleic acid molecules contained in the sample to be studied then become bound to the scavenger molecules, so that double-stranded hybrid molecules are formed. The hybridization event is thereupon detected by means of a signal caused by the redox-active label.

A biosensor disclosed here for detecting nucleic acids has at least two nucleic acid immobilization units and an electrical detection circuit. The at least two nucleic acid immobilization units of the biosensor are electrically conductive and electrically insulated from one another. In the biosensor, the at least two nucleic acid immobilization units are provided with first nucleic acid molecules acting as scavenger molecules, the first nucleic acid molecules being present as single-stranded molecules and being capable of binding second nucleic acid molecules to be detected. The first single-stranded nucleic acid molecules acting as scavenger molecules are provided with a redox-active label which can generate a detectable signal. The electrical detection circuit is configured in such a way that it detects the hybridization event of the nucleic acid molecules with the scavenger molecules by means of the label.

The device for detecting nucleic acids has a biosensor, which has at least two nucleic acid immobilization units and an electrical detection circuit. In the biosensor, the at least two nucleic acid immobilization units are in this case electrically conductive and electrically insulated from one another. The at least two nucleic acid immobilization units are provided with first nucleic acid molecules acting as scavenger molecules. The first nucleic acid molecules are in this case present as single-stranded molecules and can bind second nucleic acid molecules to be detected. The first single-stranded nucleic acid molecules acting as scavenger molecules are provided with a redox-active label which can generate a detectable signal. The electrical detection circuit is configured in such a way that it detects the hybridization event of the nucleic acid molecules with the scavenger molecules by means of the label.

Expressed clearly, the invention is based on the fact that, in contrast to the detection methods known from [1] to [5], the immobilization of the scavenger molecules and formation of the double-stranded hybrid molecules does not take place on a continuous surface (electrode) conducting the electrical current, but instead the surface is divided into regions which are each conductive per se but are not in electrical contact with one another.

The partitioning into these regions has several advantages in the biosensor according to the invention. First, in this way each region or each position which is provided with molecules working as electron pumps is loaded only with the capacitance of an individual sensor field. Secondly, the measurement arrangement can thereby be made less susceptible to noise and more secure against interference.

In the method of the invention, this partitioning affords the advantage that an immobilization unit, or a plurality of these units, is selected for the individual detection of the signal.

These aforementioned regions of the sensor face will also be referred to here as immobilization units.

In the context of the invention, the term “immobilization unit” should be understood as meaning an arrangement which has an electrically conductive surface on which the scavenger molecules can be immobilized, i.e. to which the scavenger molecules can bind by physical or chemical interactions. These interactions include hydrophobic, hydrophilic, van der Waals or ionic (electrostatic) interactions and covalent bonds. Examples of suitable surface materials, which may be used for the at least one immobilization unit, are metals such as gold or palladium or electrically conductive polymers. In the present invention, an immobilization unit constitutes an electrode or a component of an electrode.

The immobilization on a unit may be carried out by providing the entire surface of an immobilization unit with scavenger molecules. It is, however, also possible to restrict the immobilization selectively to individual regions/points (spots) of an immobilization unit. In order to achieve the latter, the immobilization unit may be configured appropriately, for example using regions which have been chemically activated for the immobilization.

The term “redox-active label” is used here with the meaning which pertains to the term “redox-active unit” in [1] to [5] and, for example, is indicated in [3] on page 9, 3rd paragraph; page 22, 4th paragraph to page 23, 3rd paragraph.

This means that, in the context of the present invention, a redox-active label is a chemical compound, group or unit made up of several molecules which has the property, under particular external conditions, of giving electrons to a suitable oxidizing agent or of taking electrons from a suitable reducing agent, that is to say it has the property, under particular external conditions, of giving electrons to a suitable electron acceptor or of taking electrons from a suitable electron donor.

A redox-active label in the context of the present invention therefore comprises the chemically inducible or photo-inducible redox-active units defined in [1] to [5] (cf. the definition of the photo-inducible redox-active unit in [3] on page 10, 2nd paragraph to page 12, 2nd paragraph, or of the chemically inducible redox-active unit on page 12, 2nd paragraph, page 13, 1st paragraph), which are (covalently) bound by at least one bond to a nucleic acid single-strand molecule acting as a scavenger molecule.

Examples of photo-inducible redox-active labels which may be used in the present method are therefore the photosynthetic bacterial reaction center (RC), cyclophanes or an at least bimolecular electron-donor/electron-acceptor complex (the latter, of course, with its meaning according to [1] to [5], see [3], page 14, 2nd paragraph, page 15, 1st paragraph). In the case of the latter complex, the electron donor and the electron acceptor of the bimolecular electron-donor/electron-acceptor complex may be a charge transfer complex or a transition metal complex.

Examples of chemically inducible redox-active labels are therefore the cytochrome bc complex, the cytochrome c₂ complex of the bacteria driving photosynthesis or chemically inducible at least bimolecular electron-donor/electron-acceptor complexes such as suitable cyclophanes (see [3], page 31, 2nd paragraph).

In general, the signal generated by the redox-active label is used for detecting nucleic acids in the method being described here. The detection is preferably carried out by measuring a current flow, a resistance or a conductivity.

In a preferred embodiment of the method, an immobilization unit is selected for individual detection of the signal.

The term nucleic acids is intended here to mean DNA molecules, RNA molecules, PNA molecules as well as shorter fragments such as oligonucleotides with, for example, from 10 to 40 base pairs (bp). The nucleic acids may be double-stranded, but may also have at least single-stranded regions or be present as single strands, for example as a result of prior thermal denaturing (strand separation) for their detection. The sequence of the nucleic acids to be detected may in this case be at least partially or fully given, that is to say known.

If DNA molecules (nucleic acids or oligonucleotides) with a predetermined nucleotide sequence are being detected by the method described here, then they are preferably detected in the single-stranded form, i.e. before the detection they are optionally converted into single strands by denaturing as explained above. In this case, DNA probe molecules with a sequence complementary to the single-stranded region are then preferably used as scavenger molecules. The DNA probe molecules may in turn comprise oligonucleotides or even longer nucleotide sequences, so long as these do not form the intermolecular structures which prevent hybridization of the probe molecules with the nucleic acid to be detected.

In one configuration of the method—and therefore in a refinement of the method known from [1] to [5], unbound DNA probe molecules are removed from the at least two immobilization units after having been brought in contact with a sample to be studied. In this way, any background current due to single-stranded marked probe molecules can at least be reduced. The removal is advantageously carried out by an enzyme with nuclease activity being brought in contact with the immobilization unit.

At least one of the following substances may is used as the enzyme with nuclease activity for the removal:

mung bean nuclease,

nuclease P1,

nuclease S1, or

DNA polymerases which are capable of degrading single-stranded DNA owing to their

5′->3′ exonuclease activity or their

3′->5′ exonuclease activity.

In a preferred embodiment of the invention, the immobilization units comprise gold or consist of gold.

In the method, in a further configuration, the at least two immobilization units are arranged on a semiconductor chip. Such a semiconductor chip is preferably a CMOS chip.

It should be pointed out that it is of course possible to detect not only a single type of nucleic acid in a single measurement run with the present invention. Rather, a plurality of nucleic acids may be detected simultaneously or successively. To this end, a plurality of types of scavenger molecules, each of which has a (specific) binding affinity for a particular nucleic acid to be detected, may be bound on the immobilization units, and/or it is possible to use a plurality of immobilization units, only one type of scavenger molecule being bound on each of these units. In these multiple determinations, a label which can be discriminated from the other labels will preferably be used for each nucleic acid to be detected, for example so as to avoid undesired side reactions.

So that it can be used in such a multiple determination, the biosensor described here preferably has a plurality of, i.e. more than two, nucleic acid immobilization units in a regular arrangement.

In an advantageous configuration of the biosensor, the electrical detection circuit is integrated in a semiconductor chip. This has the advantage that the overall measurement arrangement can thereby be simplified and a higher measurement sensitivity can be reached.

A further simplification is obtained in the biosensor by a configuration in which the immobilization units are arranged on the semiconductor chip.

In principle, any suitable semiconductor component can be used as the semiconductor chip in the biosensor.

A transistor chip is a preferably used, which may be a CMOS chip.

In a refinement of the biosensor, the electrical detection circuit has a preamplifier for preamplifying the detected signal for each immobilization unit.

In another configuration, the electrical detection circuit has selection electronics for individual selection of at least one immobilization unit. This has the advantage, on the one hand, that positioning problems during the irradiation of the sensor with light, which is necessary for a photo-inducible measurement, are thereby avoided since the position selection, i.e. which sensor field/which immobilization unit is activated, takes place electronically. In particular, this allows the biosensor to be configured as a so-called “handheld device” or permits (mobile) use, for example in medical practices, hospitals, emergency or intensive medicine or in the “home-care field”. On the other hand, the selection electronics has the advantage that the crosstalk, i.e. the interfering effect of unselected sensor positions, can be fully suppressed. The maximum number of operable sensor positions is no longer limited by the crosstalk.

In a further embodiment of the biosensor, the electrical detection circuit has an analog/digital converter for converting the detected signal for each immobilization unit. An interface to external electronics can be thereby configured digitally and is therefore very unsusceptible to electromagnetic interference.

As a further embodiment, the electrical detection circuit has an evaluation unit for the evaluation of the detected signal for each immobilization unit. The term evaluation unit is in this case intended to mean a unit which processes an incoming measurement signal, for example by adding the signal to another signal already detected by the unit or subtracting it therefrom, storing the detected signal, comparing it with other signals, and thereby generating and optionally displaying information about whether a hybridization event has taken place. “On-chip signal processing” is hence made possible by this evaluation unit.

In another configuration, the electrical evaluation unit for each immobilization unit has a unit for adding up the charge quantity imparted to the respective immobilization unit, i.e. an integrator.

Exemplary embodiments of the invention are represented in the figures and will be explained in more detail below.

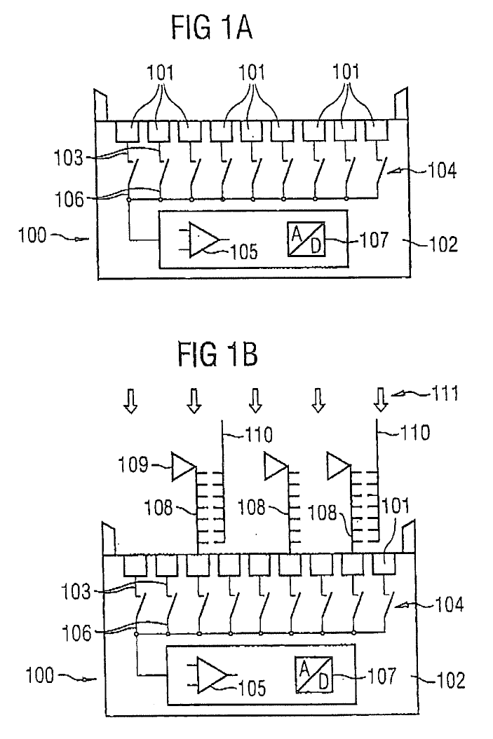

FIGS. 1a and 1b show a biosensor of the invention in different method states;

FIGS. 2a to 2d show the method of detecting nucleic acids known from [1] to [5], and a biosensor known from [1] to [5];

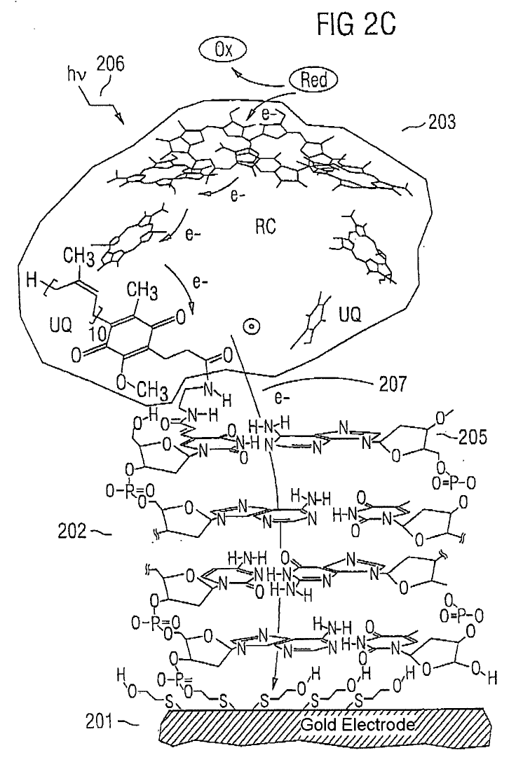

FIG. 3 shows a device with a biosensor being described here;

FIG. 1 shows a sectional view of a biosensor 100 according to an exemplary embodiment of the biosensor being described here.

FIG. 1a shows the biosensor 100 with immobilization units 101, which are arranged on an insulator layer 102 made of insulator material.

The immobilization units 101 are connected via electrical terminals 103 to an electrical detection circuit 104. The immobilization units 101 are made of gold.

The electrical detection circuit 104 of the biosensor 100 has a preamplifier 105 for amplifying the detected signal for each immobilization unit, selection electronics 106 for individually selecting at least one immobilization unit as well as an analog/digital converter 107 for converting the detected signal for each immobilization unit.

The biosensor 100 may be obtained in a two-stage method, in which it is firstly manufactured by means of a standard CMOS production method and a gold layer is subsequently applied to form the immobilization units on the chip.

Single-stranded DNA probe molecules 108, which have redox-active labels 109, are applied on the immobilization units 101 (FIG. 1b). This label 109 may, for example, be a photosynthetic bacterial reaction center and, as described in [3] on page 50, 4th paragraph to page 52, 1st paragraph, interconnected with the probe molecules (cf. also FIG. 2c).

In order to detect nucleic acids, the biosensor is brought in contact with a sample to be studied, for instance an electrolyte (not shown).

FIG. 1b shows the biosensor 100 in the event that DNA strands 110 having a predetermined nucleotide sequence which is complementary to the sequence of the DNA probe molecules 108 are contained in the electrolyte.

In this case, the DNA strands 110 complementary to the DNA probe molecules 108 hybridize with the DNA probe molecules 108, which are applied on the immobilization units 101.

As can be seen from FIG. 1b, the result after hybridization has taken place is that there are hybridized molecules on the units 101, i.e. double-stranded DNA molecules are immobilized there.

In a further stage, hydrolysis of single-stranded DNA probe molecules 108 on the units 101 may be brought about optionally by means of a biochemical method, for example by adding DNA nucleases to the electrolyte (cf. FIG. 1b).

In this case, the selectivity of the degrading enzyme for single-stranded DNA needs to be taken into account. If the enzyme selected for degrading the unhybridized DNA single strands does not have this selectivity, then the nucleic acid present as double-stranded DNA which is to be detected may possibly be (undesirably) degraded as well, which would lead to vitiation of the measurement result.

After removal of the single-stranded DNA probe molecules, only the hybrids of the DNA molecules 110 to be detected and the first DNA probe molecules 108 complementary to them are present.

For example, in order to remove the unbound single-stranded DNA probe molecules 108 on the immobilization units 101, one of the following substances may be added:

mung bean nuclease,

nuclease P1, or

nuclease S1.

DNA polymerases which, owing to their 5′->3′ exonuclease activity or their 3′->5′ exonuclease activity, are capable of degrading single-stranded DNA may also be used for this purpose.

A light source which is not shown (for example a laser) is then used to irradiate light, symbolized by arrows 111, with a wavelength which is suitable for stimulating the labels 109, for example a bacterial reaction center. This gives rise to a photo-induced charge separation inside the cofactors of the reaction center and to an intermolecular electron transfer.

If a suitable potential is applied to the immobilization units 101 (determined by the selection electronics), transfer of an electron takes place from the double-stranded hybrid molecules to the units 101, that is to say a current flow which is detected by the electrical detection circuit 104.

In this way, the presence of the DNA molecules 110 is determined. The use of the biosensor 100 described here allows individual (and position-resolved) detection of one or more immobilization units and, besides an increased measurement sensitivity, provides a significant simplification of the overall measurement arrangement.

FIG. 3 shows a device 300 for detecting nucleic acids, which has a biosensor 301 constructed in accordance with the biosensor according to Exemplary Embodiment 1. This means that the biosensor 301 has immobilization units 302, which are arranged on an insulator layer 303 made of insulator material.

The immobilization units 302 are made of gold and are connected via electrical terminals 304 to an electrical detection circuit 305. The electrical detection circuit 305 of the biosensor 301 has a preamplifier 306 for amplifying the detected signal for each immobilization unit, selection electronics 307 for individually selecting at least one immobilization unit as well as an analog/digital converter 308 for converting the detected signal for each immobilization unit.

The device 300 has a support 309, which holds the sensor 301 and can be moved on the sensor, for example for sample preparation or the application of scavenger molecules. A sample to be studied, for example a liquid analyte 310, is furthermore applied on the sensor.

The device 300 also has a light source 311 with which light symbolized by arrows 312 can be irradiated onto the sensor. The light source 312 may likewise be mobile. The device 300 lastly also has a control unit (not shown) and, for example, liquid delivery means with which it is possible to automate an experimental procedure with, for example, application of the scavenger molecules, application of solutions to be studied, removal of the solutions and the like.

The term “base-stacking perturbations” refers to any event that causes a perturbation in base-stacking such as, for example, a base-pair mismatch, a protein binding to its recognition site, or any other entities that form oligonucleotide adducts.

The term “denaturing” refers to the process by which strands of oligonucleotide duplexes are no longer base-paired by hydrogen bonding and are separated into single-stranded molecules. Methods of denaturation are well known to those skilled in the art and include thermal denaturation and alkaline denaturation.

The term “hybridized” refers to two nucleic acid strands associated with each other which may or may not be fully base-paired.

The term “intercalative moieties” refers to planar aromatic or heteroaromatic moieties that are capable of partial insertion and stacking between adjacent base pairs of double-stranded oligonucleotides. These moieties may be small molecules or part of a larger entity, such as a protein. Within the context of this invention the intercalative moiety is able to generate a response or mediate a catalytic event.

The term “mismatches” refers to nucleic acid bases within hybridized duplexes which are not 100% complementary. A match includes any incorrect pairing between the bases of two nucleotides located on complementary strands of DNA that are not the Watson-Crick base-pairs A:T or G:C. The lack of total homology may be due to deletions, insertions, inversions, substitutions or frameshift mutations.

The term “mutation” refers to a sequence rearrangement within DNA. The most common single base mutations involve substitution of one purine or pyrimidine for the other (e.g., A for G or C for T or vice versa), a type of mutation referred to as a “transition”. Other less frequent mutations include “transversions” in which a purine is substituted for a pyrimidine, or vice versa, and “insertions” or “deletions”, respectively, where the addition or loss of a small number (1, 2 or 3) of nucleotides arises in one stand of a DNA duplex at some stage of the replication process. Such mutations are also known as “frameshift” mutations in case of insertion/deletion of one of two nucleotides, due to their effects on translation of the genetic code into proteins. Mutations involving larger sequence rearrangement also may occur and can be important in medical genetics, but their occurrences are relatively rare compared to the classes summarized above.

The term “nucleoside” refers to a nitrogenous heterocyclic base linked to a pentose sugar, either a ribose, deoxyribose, or derivatives or analogs thereof. The term “nucleotide” relates to a phosphoric acid ester of a nucleoside comprising a nitrogenous heterocyclic base, a pentose sugar and one or more phosphate or other backbone forming groups; it is the monomeric unit of an oligonucleotide. Nucleotide units may include the common bases such as guanine (G), adenine (A), cytosine (C), thymine (T, or derivatives thereof. The pentose sugar may be deoxyribose, ribose, or groups that substitute therefore.

The terms “nucleotide analog”, “modified base”, “base analog”, or “modified nucleoside” refer to moieties that function similarly to their naturally occurring counterparts but have been structurally modified.

The terms “oligonucleotide” or “nucleotide sequence” refers to a plurality of joined nucleotide units formed in a specific sequence from naturally occurring heterocyclic bases and pentofuranosyl equivalent groups joined through phosphorodiester or other backbone forming groups.

The terms “oligonucleotide analog” or “modified oligonucleotides” refer to compositions that function similarly to natural oligonucleotides but have non-naturally occurring portions. Oligonucleotide analogs or modified oligonucleotides may have altered sugar moieties, altered bases, both altered sugars and bases or altered inter-sugar linkage; which are known for use in the art.

The terms “redox-active moiety” or “redox-active species” refers to a compound that can be oxidized and reduced, i.e. which contains one or more chemical functions that accept and transfer electrons.

The term “redox protein” refers to proteins that bind electrons reversibly. The simplest redox proteins, in which no prosthetic group is present, are those that use reversible formation of a disulfide bond between to cysteine residues, as in thioredoxin. Most redox proteins however use prosthetic groups, such as flavins or NAD. Many use the ability of iron or copper ions to exist in two different redox states.

The present invention provides a highly sensitive and accurate method based on an electrochemical assay using intercalative, redox-active species to determine the presence and location of a singe or multiple base-pair mismatches. Briefly, the system is comprised of (i) a reagent mixture comprising an electrode-bound oligonucleotide duplex to which an intercalative, redox-active moiety is associated and (ii) means for detecting and quantitating the generated electrical current or charge as an indication for the presence of a fully base-paired versus a mismatch containing duplex. The present invention is particularly useful in the diagnosis of genetic diseases that arise from point mutations. For example, many concerns can be traced to point mutations in kinases, growth factors, receptors binding proteins and/or nuclear proteins. Other diseases that arise from genetic disorders include cystic fibrosis, Bloom's syndrome, thalassemia and sickle cell disease. In addition, several specific genes associated with cancer, such as DCC, NF-1, RB, p53, erbA and the Wilm's tumor gene, as well as various oncogenes, such as abl, erbB, src, sis, ras, fos, myb and myc have already been identified and examined for specific mutations.

The present invention provides methods for detecting single or multiple point mutations, wherein the oligonucleotide duplex the redox-active species is adsorbed and therefore continuously exposed to an electrode whose potential oscillates between a potential sufficient to effect the reduction of said chemical moiety and a potential sufficient to effect the oxidation of the chemical moiety. This method is preferred over other methods for many reasons. Most importantly, this method allows the detection of one or more mismatches present within an oligonucleotide duplex based on a difference in electrical current measured for the mismatch-containing versus the fully base-paired duplex. Thus the method is based on the differences in base-stacking of the mismatches and is independent of the sequence composition of the hybridized duplex, as opposed to existing methods that depend on thermodynamic differences in hybridization. Furthermore, this method is nonhazardous, inexpensive, and can be used in a wide variety of applications, alone or in combination with other hybridization-dependent methods.

One particular aspect of the invention relates to the method for sequential detection of mismatches within a number of nucleic acid samples which comprises the following steps. At least one strand of a nucleic acid molecule is hybridized under suitable conditions with a first nucleic acid target sequence forming a duplex which potentially contains a mismatch, and wherein one of the nucleic acids is derivatized with a functionalized linker. This duplex is then deposited onto an electrode or an addressable multielectrode array forming a monolayer. An intercalative, redox-active species (e.g., daunomycin) is noncovalently adsorbed (or crosslinked, if desired) onto this molecular lawn, and the electrical current or charge generated is measured as an indication of the presence of a base pair mismatch within the adsorbed oligonucleotide complex. Subsequent treatment of the duplexes containing the intercalative, redox-active species under denaturing conditions allows separation of the complex, yielding a single-stranded monolayer of oligonucleotides which can be rehybridized to a second oligonucleotide target sequence. The steps of duplex formation, adsorption of the intercalative, redox-active species, measurement of the electrical current or charge, and denaturation of the complex to regenerate the single-stranded oligonucleotides may be repeated as often as desired to detect in a sequential manner genetic point mutations in a variety of oligonucleotide probes.

The charges passed at each of the electrodes is measured and compared to the wild-type, i.e. fully base-paired, sequences. Electrodes with attenuated signals correspond to mutated sequences, while those which exhibit no change in electrical current or charge are unmutated. Furthermore, the intensity of the signal compared to the wild-type sequence not only reports the presence of the mismatch but also describes the location of the disruption within the analyzed duplex.

Another aspect of the invention relates to the method of detecting mutations utilizing electrolysis. Briefly, the modification of electrode surfaces with oligonucleotide duplexes provides a medium that is impenetrable by negatively charged species due to the repulsion by the high negative charge of oligonucleotides. However, electrons can be shuttled through the immobilized duplexes to redox-active intercalators localized on the solvent-exposed periphery of the monolayer, which in turn can catalytically reduce these negatively charged species. More specifically, this electrocatalytic method comprises the following steps. At least one strand of a nucleic acid molecule is hybridized under suitable conditions with a first nucleic acid target sequence forming a duplex which potentially contains a mismatch, and wherein one of the nucleic acids is derivatized with a functionalized linker. This duplex is then deposited onto an electrode or a multielectrode array forming a monolayer. The assembly is immersed into an aqueous solution containing both an intercalative, redox-active species (e.g., methylene blue) and a non-intercalative, redox-active species (e.g., ferricyanide). The electrical currents or charges corresponding to the catalytic reduction of ferricyanide mediated by methylene blue are measured for each nucleic acid-modified electrode and compared to those obtained with wild-type, i.e. fully base-paired sequences. Subsequent treatment of the duplexes under denaturing conditions allows separation of the complex, yielding a single-stranded monolayer of oligonucleotides which can be rehybridized to a second oligonucleotide target sequence. The steps of duplex formation, measurement of the catalytically enhanced electrical current or charge, and denaturation of the complex to regenerate the single-stranded oligonucleotides may be repeated as often as desired to detect in a sequential manner genetic point mutations in a variety of oligonucleotide probes. This particular method based on electrocatalysis at oligonucleotide-modified surfaces is extremely useful for systems where attenuated signal resulting from the presence of mismatches are small. The addition of a noninterlative electron acceptor amplifies the signal intensity, and allows more accurate measurements. This approach may be particularly useful to monitor assays based on redox-active proteins which bind to the oligonucleotide-modified surface, but are not easily oxidized or reduced because the redox-active center is not intercalating.

The present invention further relates to the nature of the redox-active species. These species have a reduced state in which they can accept electron(s) and an oxidized state in which they can donate electron(s). The intercalative redox-active species that are adsorbed or covalently linked to the oligonucleotide duplex include, but are not limited to, intercalators and nucleic acid-binding proteins which contain a redox-active moiety.

An intercalator useful for the specified electrochemical assays is an agent or moiety capable of partial insertion between stacked base pairs in the nucleic acid double helix. Examples of well-known intercalators include, but are not limited to, phenanthridines (e.g., ethidium), phenothiazines (e.g., methylene blue), phenazines (e.g. phenazine methosulfate), acridines (e.g., quinacrine), anthraquinones (e.g.,

The following publications are cited in this document:

[1] DE 199 01 761 A1

[2] DE 199 26 457 A1

[3] WO 00/42217 A1

[4] DE 199 21940 A1

[5] WO 00/31101 A1

[6] U.S. Pat. No. 5,653,939

[7] U.S. Pat. No. 6,017,696

### List of references

100 biosensor

101 immobilization units

102 insulator layer

103 electrical terminals

104 electrical detection circuit

105 preamplifier

106 selection electronics

107 analog/digital converter

108 DNA probe molecules

109 redox-active label

110 DNA strands

200 sensor

201 conductive surface layer

202 single-stranded nucleic acid molecules

203 redox-active label

204 electrical terminal

205 DNA strands

206 light symbolized by arrow

207 arrow

208 test sites

209 support material

210 analyte

300 device for detecting nucleic acids

301 sensor

302 immobilization units

303 insulator layer

304 electrical terminals

305 electrical detection circuit

306 preamplifier

307 selection electronics

308 analog/digital converter

309 support

310 analyte

311 light source

312 light symbolized by arrow

1. A method for detecting nucleic acids by means of at least two nucleic acid immobilization units,
in which the at least two nucleic acid immobilization units are electrically conductive and electrically insulated from one another,
in which the at least two nucleic acid immobilization units are provided with first nucleic acid molecules acting as scavenger molecules, the first nucleic acid molecules being present as single-stranded molecules and being capable of binding second nucleic acid molecules to be detected, and the first single-stranded nucleic acid molecules acting as scavenger molecules being provided with a redox-active label which can generate a detectable signal,
in which a sample to be studied is brought in contact with the at least two nucleic acid immobilization units, wherein the sample to be studied may contain the second nucleic acid molecules to be detected,
in which second nucleic acid molecules contained in the sample to be studied become bound to the scavenger molecules, so that double-stranded hybrid molecules are formed,
in which the second nucleic acid molecules are detected by means of a signal caused by the redox-active label, by using an electrical detection circuit integrated in a semiconductor chip, and
in which an immobilization unit is selected for individual detection of the signal.

2. The method as claimed in claim 1, in which the signal generated by the redox-active label is detected by measuring a current flow, a resistance or a conductivity.

3. The method as claimed in claim 2, in which the redox-active label is a photo-inducible redox-active label or a chemically inducible redox-active label.

4. The method as claimed in claim 3, in which the photo-inducible redox-active label is a photosynthetic bacterial reaction center, a cyclophane or an at least bimolecular electron-donor/electron-acceptor complex.

5. The method as claimed in claim 4, in which the electron donor and the electron acceptor of the bimolecular electron-donor/electron-acceptor complex is a charge transfer complex or a transition metal complex.

6. The method as claimed in one of claims 1 to 5, in which DNA molecules, RNA molecules or PNA molecules are detected as the nucleic acids.

7. The method as claimed in claim 6,
in which DNA or RNA single strands with a predetermined nucleotide sequence are detected as the nucleic acid molecules, and
in which DNA probe molecules with a nucleotide sequence complementary to the predetermined nucleotide sequence are used as scavenger molecules.

8. The method as claimed in claim 7, in which unbound DNA probe molecules are removed from the at least two immobilization units.

9. The method as claimed in claim 8, in which an enzyme with nuclease activity is brought in contact with the immobilization unit in order to remove unbound DNA probe molecules.

10. The method as claimed in claim 9, in which at least one of the following substances is used as the enzyme with nuclease activity:
mung bean nuclease,
nuclease P1,
nuclease S1, or
DNA polymerases which are capable of degrading single-stranded DNA owing to their
5′->3′ exonuclease activity or their
3′->5′ exonuclease activity.

11. The method as claimed in one of claims 1 to 10, in which the immobilization units comprise gold.

12. The method as claimed in one of the preceding claims, in which the at least two immobilization units are arranged on a semiconductor chip.

13. The method as claimed in claim 12, in which the semiconductor chip is a CMOS chip.

14. A biosensor for detecting nucleic acids with at least two nucleic acid immobilization units and with an electrical detection circuit,
in which the at least two nucleic acid immobilization units are electrically conductive and electrically insulated from one another,
in which the at least two nucleic acid immobilization units are provided with first nucleic acid molecules acting as scavenger molecules, the first nucleic acid molecules being present as single-stranded molecules and being capable of binding second nucleic acid molecules to be detected, and the first single-stranded nucleic acid molecules acting as scavenger molecules being provided with a redox-active label which can generate a detectable signal, and
in which the electrical detection circuit is configured in such a way that the detection circuit detects nucleic acid molecules, which have bound to the scavenger molecules, by means of the label, and
in which the electrical detection circuit has selection electronics for individual selection of at least one immobilization unit, and in which the electrical detection circuit is integrated in a semiconductor chip.

15. The biosensor as claimed in claim 14, which has a plurality of nucleic acid immobilization units in a regular arrangement.

16. The biosensor as claimed in claim 15, in which the immobilization units are arranged on the semiconductor chip.

17. The biosensor as claimed in claim 16, in which the semiconductor chip is a CMOS chip.

18. The biosensor as claimed in one of claims 14 to 17, in which the electrical detection circuit has a preamplifier for preamplifying the detected signal for each immobilization unit.

19. The biosensor as claimed in one of claims 14 to 18, in which the electrical detection circuit has an analog/digital converter for converting the detected signal for each immobilization unit.

20. The biosensor as claimed in one of claims 14 to 19, in which the electrical detection circuit has an evaluation unit for the detected signal for each immobilization unit.

21. The biosensor as claimed in one of claims 14 to 20, in which the electrical evaluation unit for each immobilization unit has a unit for adding up the charge quantity imparted to the respective immobilization unit.

22. A device for detecting nucleic acids with a biosensor, which has at least two nucleic acid immobilization units and an electrical detection circuit, in the biosensor:
the at least two nucleic acid immobilization units being electrically conductive and electrically insulated from one another,
the at least two nucleic acid immobilization units being provided with first nucleic acid molecules acting as scavenger molecules, the first nucleic acid molecules being present as single-stranded molecules and being capable of binding second nucleic acid molecules to be detected, and the first single-stranded nucleic acid molecules acting as scavenger molecules being provided with a redox-active label which can generate an electrochemically detectable signal, and
the electrical detection circuit being configured in such a way that the detection circuit detects nucleic acid molecules, which have bound to the scavenger molecules, by means of the label, and
in which the electrical detection circuit has selection electronics for individual selection of at least one immobilization unit, and in which the electrical detection circuit is integrated in a semiconductor chip.
